(19) United States
(12) Patent Application Publication
(10) Pub. No.: US 2023/0270908 A1
(43) Pub. Date: Aug. 31, 2023

(54) APPARATUS FOR SEDIMENTATION AND OXIDATION OF FINE DUST AND AEROSOLS AND ORGANIC LOADS SUCH AS VIRUSES IN A DEVICE, AND FOR ELECTROSTATIC AND ELECTROCHEMICAL OR PHOTOCHEMICAL PROCESSESING OF THE AIR SUPPLIED BY THE DEVICE TO A ROOM FOR ANALOG PROCESSES IN THE ROOM

(71) Applicant: PROACTIVEAIR GMBH, Herrenberg-Gueltstein (DE)

(72) Inventor: Jörg Lehmann, Kesselsdorf bei Dresden (DE)

(21) Appl. No.: 18/014,338

(22) PCT Filed: Feb. 26, 2021
(86) PCT No.: PCT/EP2021/054795

§ 371 Date: Jan. 3, 2023

(30) Foreign Application Priority Data
Jul. 3, 2020 (DE) 20 2020 002 865.7

(51) Int. Cl. A61L 9/014; F24F 8/40; F24F 8/22; F24F 8/30; F24F 8/192; F24F 8/167; A61L 9/20; A61L 9/22; A61L 9/12; A61L 9/04; B03C 3/016; B03C 3/32; B01D 53/86; B01D 53/32; B01D 53/75

(52) U.S. Cl. CPC A61L 9/014; F24F 8/40; F24F 8/22; F24F 8/30; F24F 8/192; F24F 8/167; A61L 9/20; A61L 9/22; A61L 9/12; A61L 9/046; B03C 3/016; B03C 3/32; B01D 53/8687; B01D 53/323; B01D 53/75; A61L 2209/14; A61L 2209/12; B01D 2251/104; B01D 2257/90; B01D 2257/91; B01D 2255/20707; B01D 2258/06; B01D 2259/804; B01D 2259/4508

(57) ABSTRACT

The present invention relates to a device for keeping room air clean and a corresponding method.

The invention concerns a device for keeping the air clean according to claim 1 and a corresponding method according to claim 9.

Air recirculation and air conditioning devices that clean the air via ionization and ozone formation in individual devices have been known for more than 50 years. Air conditioning units are also state of the art, which emit and introduce ions and ozone into the room in order to reduce and oxidize particles, odors and germs on site. They are so-called stand-alone devices, which are characterized by the fact that they are transportable, have their own fan and suck in the room air, treat it and feed it back into the room to improve the room air quality. These now have treatment combinations such as seen in direction of air flow a pre-filter, formaldehyde filter, ozone filter subject to pressure loss, TiO₂ filter, which should reduce germs and organic pollution in connection with downstream UV-LEDs and a suction-side fan. In the individual treatment stages, the loads are reduced in sequence.

Other systems generate ozone and negative small ions for the room in connection with UV-C discharge lamps and downstream ionization units as well as a dust collector.

The disadvantage of all previously used and known systems is that there is no residue-free decomposition of germs and bacteria, most VOC (volatile organic compounds -volatile hydrocarbons) and organic components in the ventilation system itself. As a rule, these substances and incomplete decomposition products are deposited on surfaces that have to be cleaned regularly, or in adsorption or absorption storage devices such as e.g. B. Activated carbon, which also serve as a residual ozone destroyer and which must be replaced regularly, is deposited.

In addition, the generation and introduction of ozone into the room for the treatment of the room as a source of emissions and the main source of infection with viruses and bacteria is not secured as required. A room volume is usually specified for which the devices can be used. This is mainly done to prevent too much ozone entering the room for health reasons, the max. ozone load should not permanently exceed 50 μg/m³. This means that these devices are severely restricted in their use. Since the ozone generating elements either produce a stable amount of ozone in g/h in the device or regulated ozone elements are used which, due to the half-life of ozone, do not increase the ozone in the room in a controllable manner, these devices and systems can only be used to a limited extent. With regard to the killing of germs and the degradation of organic components in the form of oxidation, such systems are limited. More than 90% of germs are broken down in a short time only if the ozone load generated is well above the health-threatening 50 μg/m³. In addition, the germs and bacteria must be deposited on surfaces, since even with the high ozone loads of over 50 μg/m³ there is not a sufficient ozone concentration for oxidative processes in the air.

Even combinations of ozone from UV-C with a wavelength of 180 nm and UV-C light with a wavelength of 253.7 nm do not bring the desired parameters for the room, since UV-C light via discharge lamps cannot be controlled and when several UV-C lamps are connected in cascade with the switching off of some lamps, in addition to the reduction of ozone, the desired UV-C disinfection performance at 253.7 nm is no longer given due to the lower radiation density.

Thus, with the existing air cleaning systems, the disinfection performance is not completely guaranteed, especially with viral and bacterial loads and their residual products and is a problem with highly contagious germs such as e.g. viruses continue to pose a significant risk.

The objective technical task of the invention is to provide an air cleaning component for primarily stand-alone devices, i.e. portable devices that are operated in air recirculation mode in the room, which safely and reliably kills sucked-in organic pollution, in particular viruses and bacteria as well as other germs, in the device and thereby enabling a constant loading of the room air with ozone and ions.

According to the invention, the object is achieved by a device and a method according to claims 1 and 9.

It is provided according to an embodiment of the invention that in the air duct in the air flow direction, the air flow is first irradiated by means of UV-C radiation, preferably with a wavelength of >230 nm, primarily 253.7 nm, in such a way that the radiation in addition to the air flow and the organic Impurities such as viruses are also captured by a downstream collector of an electro-precipitation unit in the area. At the same time, ozone can be supplied to the air flow or formed from the air flow, with the amount of ozone preferably being adjusted in such a way that bacteria, viruses or other germs and odors can be oxidized. After the UV-C generator unit and the ozone unit, there is an electrostatic precipitator, preferably in the direction of the air flow, consisting of discharge electrodes and a collector, on which the charged particles and aerosols, preferably <2.5 μm, are separated. On the surface of the collector and in the air flow, germs and viruses are inactivated by the UV-C light, preferably with a wavelength >230 nm, whose radiation, measured in J/m², can be set to the maximum air volume. The ozone in the air flow and generated in the device means that the germs deposited on the surfaces of the collector are primarily oxidized to CO₂ and H₂O and the collector surfaces are thus almost completely free of organic contamination. The air, which has now been cleaned of aerosols, fine dust and particles as well as viruses, bacteria, germs, fungi and other organic pollution, but is still contaminated with residual ozone from our own ozone production or from external sources via the sucked-in air, can be treated with a residual ozone destroyer in such a way that that there are only ozone levels below the health risk limit of 100 μg/m³. The residual ozone can be destroyed by common catalysts such as activated carbon or zeolites, but also by UV light, for example with a wavelength of >230 nm, which is known to break down ozone. On the outlet side, the aerosol-free and fine dust-free air is ionized and, if necessary, mixed with a quantity of ozone defined for the size of the room in such a way that a minimum quantity of small ions of 500/cm³ and a maximum quantity of ozone of 100 μg/m³ can be measured directly at the device outlet and thus in the room can have a deodorizing effect. The generation can take place via an actuating mechanism in such a way that either only ions, and here negative or positive ions, only ozone or separately adjustable or controllable ions and ozone are generated and released into the room air either manually or via measuring sensors and a control circuit. The concentration of the sucked-in ozone can preferably be measured and the total amount of ozone supplied to the room can be adjusted accordingly via a control circuit.

The advantage of the invention consists in the reliable killing of all germs, viruses and bacteria outside of and in the device itself and their (almost) residue-free oxidation to CO₂ and H₂O in the device, so that no endo- or mycotoxins or other organic residues as growth bases for others germs and bacteria are present when the device is idle.

In addition, the room below, in which the ionized and ozonized air from the device is taken and which represents the actual field of germ spread by living beings via aerosols from breathing, is cleaned to over 90% of germ-carrying fine aerosols by their sedimentation. The sedimented aerosols and germs such as viruses and bacteria are oxidized on the surfaces and to a small extent in the air by means of the ozone to form CO₂ and H₂O with almost no residue. According to a preferred embodiment of the present invention, the air cleaning component, which can also be referred to as an air cleaning device, has the following structure:
Air flows through the device, which is drawn in through an opening and, after being treated, is expelled through another opening of the device. In this case, air flows in a defined direction through the device between the inlet opening and the outlet opening. In the area of the inlet opening, a device is preferably provided which is designed to decompose ozone, which is present in the sucked in room air. A source of UV radiation, in particular of a defined wavelength, preferably of the wavelength >230 nm, can be provided for this purpose, for example. The radiation intensity of the radiation source can be adapted to destroy at least the amount of ozone that the device releases into the room air at its outlet opening. In particular, this can relate to the concentration of ozone or the absolute amount of ozone. The concentration of ozone can depend on the size of the room in which the device is provided, or the volume of air constantly circulating in a closed room.

Particularly preferably, the radiation intensity of the radiation source can be adapted to a measured amount of ozone in the area of the entry opening for air into the device. This allows for a particularly energy-efficient treatment of the room air.

According to a preferred embodiment of the present invention, an electrostatic precipitator is provided in the air cleaning device. This is preferably arranged opposite the radiation source in the inlet opening of the air in such a way that a deposition surface of the electrostatic precipitator is irradiated by the radiation source. In this way, a hygienic separation of dirt particles, viruses and bacteria and their passivation or decomposition into carbon dioxide and water can be brought about in a particularly simple manner.

The device, which provides air in the area of the inlet opening for the device according to the invention, makes the air flowing through the device preferably low in ozone, particularly preferably ozone-free. According to the present invention, air is considered to be ozone-free when the ozone concentration in the measured air volume is less than 10 μg/m³, 1 μg/m³, 0.1 μg/m³ or 0.01 μg/m³.

This purified and ozone-free air is passed through another filter, for example, which is particularly suitable for removing VOCs (volatile organic compounds—volatile hydrocarbons) from the room air. A device for generating ozone and/or a device for generating negatively charged ions can be provided in front of or in a region of the outlet opening for cleaned air from the device according to the invention.

The combination of ozone decomposition in or behind the air inlet opening in the device according to the invention and ozone generation in the cleaned air before it exits the device always produces a constant ozone concentration in the room air. An accumulation of ozone in the room air volume to be cleaned is thus prevented without the need for special measuring devices for detecting the ozone concentration in the room.

An embodiment of the device for keeping the air clean against ultra-fine dust and aerosols in the room, it is presented below.

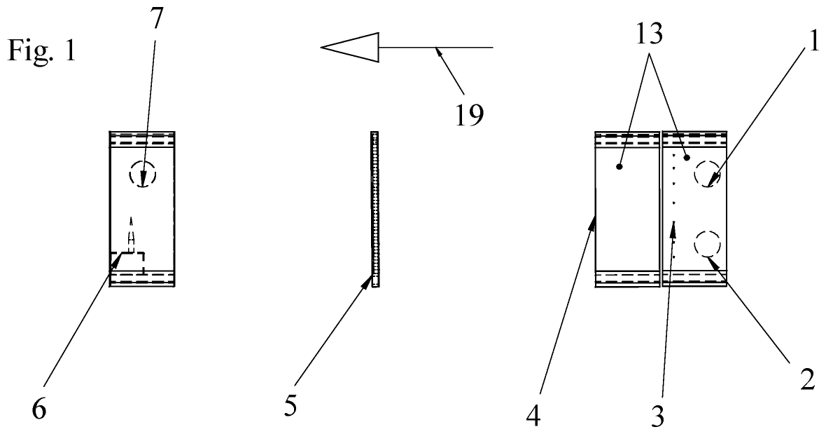

FIG. 1 the basic structure according to the invention of an air treatment unit for breaking down viruses, bacteria or other germs in the supplied air.

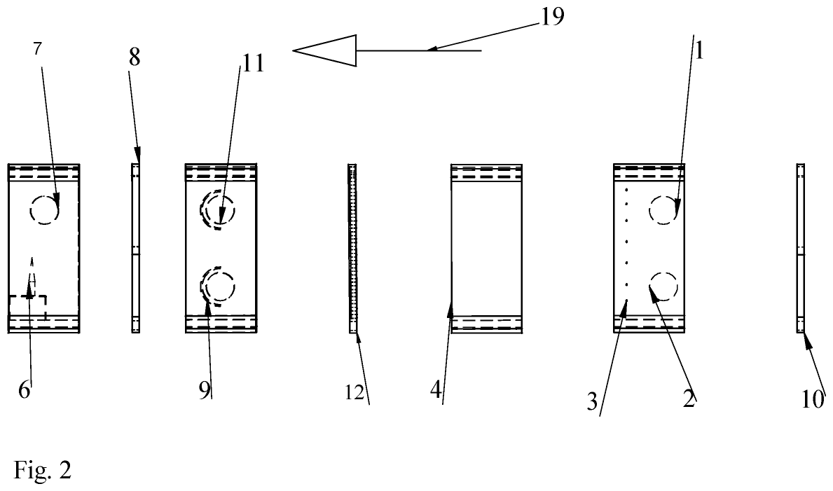

FIG. 2 the structure of the air treatment unit according to the invention with a special pressure-loss-free residual ozone destruction.

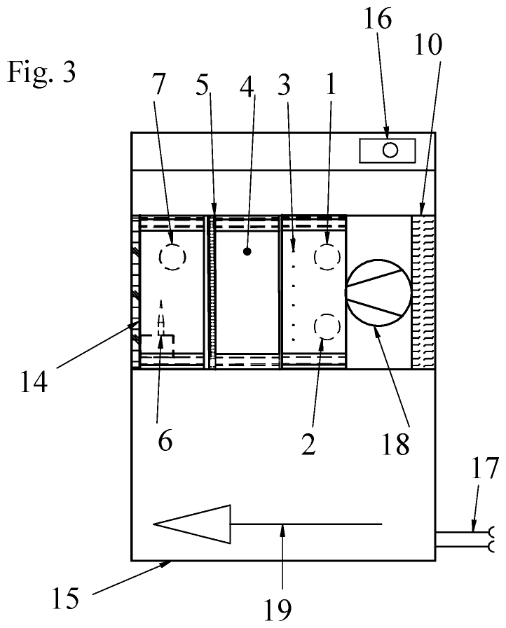

FIG. 3 the structure of the air treatment unit according to the invention in a single room air cleaner in the form of a recirculation device.

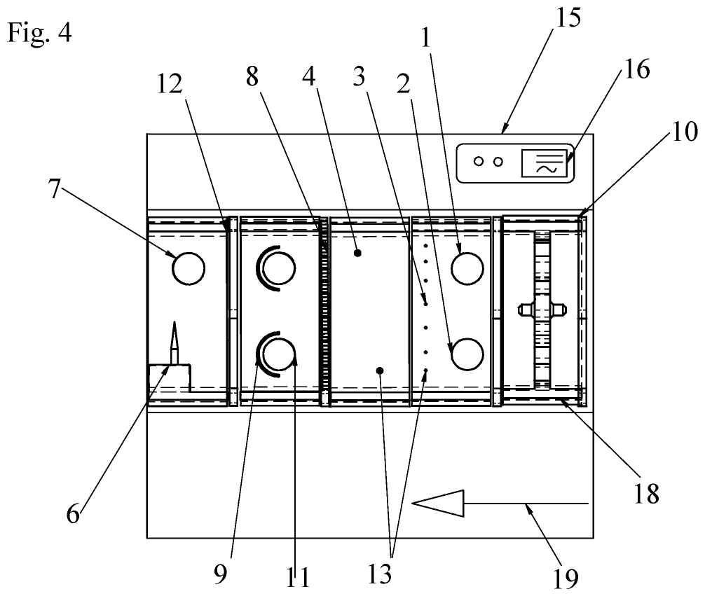

FIG. 4 the structure of the air treatment unit according to the invention in a single room air cleaner in the form of a recirculation device.

According to FIG. 1, the air flow is first passed through a first UV-C unit 1 for UV-C light emission with at least a first wavelength (e.g. >230 nm wavelength) for germ inactivation/ozone depletion and through a second UV-C Unit 2 for UV-C light emission with at least a second wavelength (z. B. <230 nm wavelength) out for ozone generation. In this case, the first unit preferably has a higher emission wavelength than the second unit, at least in its emission maximum. The second UV-C unit is optional. The first UV-C unit 1 can preferably be arranged to irradiate air flowing/being sucked into the device.

In the flow direction of the air through the device according to the invention (arrow), after the UV-C unit 1 or between the UV-C units 1, 2, the electro-separation unit 13 is preferably arranged, preferably having discharge electrodes e.g. for electro-separation 3 for electrostatically charging the air and/or or a collector 4 of the electro-separation unit for separating charged particles, in particular fine dust and aerosols also outside of the device, as well as germs, bacteria and viruses inactivated in UV-C light with a longer wavelength (preferably of >230 nm wavelength).

The UV-C unit 1 for longer wavelength UV-C light emission (>230 nm) for germ inactivation and/or the second UV-C unit 2 is/are (in the vicinity of the collector) attached and installed in such a way that the light radiation irradiates the plate surfaces of the collector 4 of the electro-deposition unit 13, i.e. preferably facing them. The ozone formed from the UV-C unit 1 oxidizes the separated organic residues primarily to form CO₂ and H₂O. Excess ozone can be broken down in an ozone filter 5 which is optionally provided downstream. The air flow, which is now free of germs, viruses and bacteria and/or fine dust, can preferably be ionized negatively or optionally positively via an ionization unit 6 before it is discharged from the device. The air can be enriched with ozone by means of an ozone generator 7, which can preferably be switched on separately and/or regulated as a function of different control variables such as air quantity or ozone concentration of the supplied air or odor pollution. The ozone generator 7, which is arranged opposite the electrostatic precipitator and/or the UV-C unit 1 downstream of the air flow, can also be designed to randomly generate a constant amount of ozone. As a result, a constant amount of ozone is released into the room air in a particularly preferred manner. Incidentally, if only the first UV-C unit 1 and the ozone generator 7 are provided, i.e. not the second UV-C unit 2, all ozone is consequently first destroyed from the sucked in air and before the air leaves the filter device, again enriched with a defined amount of ozone. As a result, the concentration of ozone in the room air can be kept constant without any ozone accumulation in the room air.

FIG. 2 shows the structure of the air treatment unit according to a preferred embodiment of the invention with a special pressure-loss-free residual ozone annihilation.

In this case, the air flow is led through an input filter 10 via a UV-C unit 1 for longer wavelength UV-C light emission (preferably >230 nm wavelength) for germ inactivation and/or via a UV-C unit 2 for UV-C Light emission with shorter wavelength (preferably <230 nm wavelength) for ozone generation as described before. After the UV-C units 1, 2, there is preferably an electro-separation unit 13, having discharge electrodes for electro-separation 3 for electrostatically charging the air and a collector of the electro-separation unit 4 for separating/collecting charged particles, in particular fine dust and aerosols as well as those germs inactivated in the UV-C light longer wavelengths (preferably >230 nm wavelength), bacteria and viruses. The UV-C unit 1 for UV-C light emission with a longer wavelength (preferably >230 nm) for germ inactivation is attached and installed in such a way that the light radiation irradiates the plate surfaces of the collector 4 of the electrodeposition unit 13. The ozone that is preferably carried along, formed from the UV-C unit 2 for UV-C light emission with a shorter wavelength (preferably <230 nm) for ozone generation, can oxidize the separated organic residues primarily to form CO₂ and H₂O. This can be followed by a low-pressure-loss privacy screen 8, which can prevent UV-C light from escaping optically from the collector 4 in the airflow direction. If the second UV-C unit is provided, excess ozone can be broken down by UV light from a UV unit 11 for UV light emission with a longer wavelength (preferably >230 nm) for ozone breakdown by irradiation. In order to prevent the UV-C light from escaping from the device, each UV-C radiator or radiation unit can be provided with a glare protection 9 in the direction of the air outlet. An additional TiO₂ catalyst for breaking down germs 12, which also oxidizes off chemical compounds, can complete the structure.

The air flow, which is now free of germs, viruses and bacteria as well as fine dust, can be ionized negatively or optionally positively via an ionization unit 6 before it is discharged from the device. The air can be enriched with ozone by means of an adjustable and/or controllable (or without) ozone generator 7 that can be switched on separately and controlled as a function of different controlled variables such as air quantity or ozone concentration of the supplied air or odor pollution.

FIG. 3 shows the structure of the air treatment unit according to the invention in a single room air cleaner in the form of a recirculation device.

On the inlet side, coarse dirt can be removed from the sucked-in air flow via an inlet filter 10. A fan 18 can then follow, which is preferably equipped with a power control. Thereafter, the air flow via a UV-C unit 1 for longer wavelength UV-C light emission (preferably >230 nm wavelength) for germ inactivation and/or via a UV-C unit 2 for shorter wavelength UV-C light emission (preferably <230 nm wavelength) for ozone generation. The UV-C units 1, 2 can be followed by the electro-separation unit 13, which has discharge electrodes for electro-separation 3 for electrostatically charging the air and/or the collector of the electrostatic precipitator unit 13 for separating charged particles, in particular fine dust and aerosols and/or the germs, bacteria and viruses inactivated in UV-C light with a wavelength of >230 nm. The UV-C unit for UV-C light emission 1 with a longer wavelength (preferably >230 nm) for germ inactivation can be attached and installed in such a way that the light radiation irradiates the plate surfaces of the collector 4 of the electrostatic precipitator unit 13, i.e. aligned in their direction is. If the second UV-10 unit is provided, the ozone carried along from it can primarily oxidize the separated organic residues to form CO₂ and H₂O. Otherwise, the first UV-C unit can at least passivate the viruses and bacteria collected on the electronic unit. Excess ozone from the second UV-C unit that is preferably provided can be broken down in the ozone filter 5 that preferably follows. The air flow, which is now free of germs, viruses and bacteria as well as fine dust (due to the electro-separation unit), can be ionized negatively or optionally positively via an ionization unit 6 before it is discharged from the device. The air can be enriched with ozone by means of an ozone generator 7 which can additionally switched on and/or can be regulated as a function of different controlled variables such as air quantity or ozone concentration of the supplied air or odor pollution. Finally, an exit grid 14 can be installed as a visual protection against the UV-C radiation and/or as a protection against accidental contact for the ozone generator 7 and the ionization unit 6. The air output, germ reduction output and the air prepared for the room via the ionization unit 6 and the ozone generator 7 with ozone and ions can be regulated or controlled in their individual parameters by means of a controller and/or regulator 16.

All built-in elements are designed to be exchangeable for cleaning, repair and maintenance purposes.

FIG. 4 shows a preferred structure of the air treatment unit in a stand-alone room air cleaner in the form of a recirculation device, preferably with an electric plug 17 and/or preferably a low-pressure-loss reduction of the ozone formed in the device. The air flow in a single device 15 for the room can be routed over an input filter 10 and/or fan 18 first via a UV-C unit 1 for UV-C light emission with a longer wavelength (preferably >230 nm wavelength) for germ inactivation and/or via a UV-C unit 2 for UV-C light emission (<230 nm wavelength) for ozone generation. The UV-C units 1, 2 are preferably followed by the electro-separation unit 13, preferably having discharge electrodes for electro-separation 3 for electrostatically charging the air and/or the collector 4 of the electro-separation unit for separating charged particles, in particular fine dust and aerosols as well as the UV Longer wavelength C-light (preferably >230 nm wavelength) inactivated germs, bacteria and viruses. The UV-C unit 1 for UV-C light emission with a longer wavelength (preferably >230 nm) for germ inactivation can be attached and installed in such a way that the light radiation irradiates the collector 4 or its plate surfaces of the electrodeposition unit, i.e. aligned with it is. The entrained ozone, formed from the UV-C unit 2 for UV-C light emission with a shorter wavelength (preferably <230 nm) for ozone generation, oxidizes the separated organic residues primarily to CO₂ and H₂O 8 follow, which can prevent the optical radiation emission of UV-C light from the collector 4 in the air direction. Excess ozone can be decomposed by UV light from a UV unit for longer wavelength UV light emission (preferably >230 nm) for ozone depletion 11 by irradiation. In order to prevent the UV-C light from breaking through from the device, each UV-C emitter or the radiation units can be provided with a glare protection 9 in the direction of the air outlet. An additional TiO₂ catalyst for breaking down germs 12, which also oxidizes chemical compounds, can complete the structure.

The air flow, which is now free of germs, viruses and bacteria as well as fine dust, can be ionized negatively or optionally positively via an ionization unit 6 before it is discharged from the device. The air can be enriched with ozone by means of a controllable and/or controllable ozone generator 7 that can be switched on separately and/or controlled as a function of different controlled variables such as air volume or ozone concentration of the supplied air or odor pollution. The individual air parameters such as air volume, ozone concentration or ion concentration can be regulated or adjusted by means of sensors and a regulation or control 16.

All assemblies are preferably of modular design and can be individually removed or replaced from the device.

The individual devices from the above-mentioned embodiments can be supplemented and/or substituted in a simple manner. Their combinations are hereby part of the disclosure of the application. According to the present invention, devices from one embodiment can also be provided in another embodiment or in any (sub)combination. This applies in particular to additional filters, catalytic converters and ventilation systems.

### REFERENCE LIST

1 UV-C unit for longer wavelength UV-C light emission (preferably >230 nm) for germ inactivation
2 UV-C unit for shorter wavelength UV-C light emission (<230 nm preferred) for ozone generation
3 Spray electrodes for electrodeposition
4 collector of the electrostatic precipitator unit
5 ozone filter
6 ionization unit/emitter
7 ozone generator
8 privacy screen
9 glare protection
10 input filter
11 UV unit for UV light emission >230 nm for ozone depletion
12 TiO₂ catalyst for breaking down germs
13 electrostatic precipitator unit
14 output grid
15 Stand alone device
16 control regulation device
17 power connection
18 Fan
19 air direction

1. Antibacterial and/or antiviral air filter device for removing ultrafine aerosols and particles <2.5 μm and for inactivating bacterial or viral contaminants contained therein comprising:
at least one UV light device (1),
an electrostatic precipitator (13),
at least one ozone-generating UV light device (7) and
an ionizer,
wherein,
an air stream to be cleaned is guidable through the air filter device, with the at least one UV light device (1) being arranged first in the direction of air flow between an inlet and an outlet for the air stream in the air filter device, which is equipped with at least one O₃-destroying and with or without an antibacterial active wavelength, wherein downstream of the UV light device (1) the electrostatic precipitator (13) is arranged, which is designed to charge the aerosols, the particles and/or the organic contaminants in the ozone-free air flow and separate them on the electrostatic precipitator, and
that downstream of the electrostatic precipitator (13) the ozone-generating UV light device (7) is provided, which is designed to emit a constant amount of ozone to the cleaned air.

2. Antibacterial air filter device according to claim 1,
wherein a catalyst for the degradation of chemical substances is provided.

3. Antibacterial air filter device according to claim 1,
wherein the UV light device (1) and a collector of the electrostatic precipitator face each other, so that the collector can be irradiated by the UV light device.

4. Antibacterial air filter device according to claim 1,
wherein the UV light device emits UV-C radiation with >230 nm, which is formed from discharge tubes or diodes or by laser light.

5. Antibacterial air filter device according to claim 1,
wherein a low-temperature plasma, dielectric barrier discharge, UV-C light with a wavelength of <230 nm or a laser are provided for the formation of ozone in an air duct.

6. Antibacterial air filter device according to claim 1,
wherein for ozone depletion in air duct catalysts are provided in addition to or instead of the UV radiation source with a wavelength >230 nm.

7. Antibacterial air filter device according to claim 1,
wherein the air exiting an air duct and fed into a room can be enriched as required by low-temperature plasma, dielectric barrier discharge, UV-C light with a wavelength of <230 nm or laser-generated ozone.

8. Antibacterial air filter device according to claim 1,
wherein all units used are interchangeably installed in a housing and/or an ionizer is provided, which is arranged downstream of the electrostatic precipitator and is provided to generate a proportion of ions from the cleaned air.

9. Air purification process with an antibacterial and/or antiviral air filter device for removing ultrafine aerosols and particles <2.5 μm and for inactivating bacterial or viral contaminants contained therein, wherein the air filter device comprises:
at least one UV light device (1),
an electrostatic precipitator (13),
at least one ozone-generating UV light device (7) and
an ionizer,
wherein,
an air stream to be cleaned is guidable through the air filter device, with the at least one UV light device (1) being arranged first in the direction of air flow between an inlet and an outlet for the air stream in the air filter device, which is equipped with at least one O₃-destroying and with or without an antibacterial active wavelength,
wherein downstream of the UV light device (1) the electrostatic precipitator (13) is arranged, which is designed to charge the aerosols, the particles and/or the organic contaminants in the ozone-free air flow and separate them on the electrostatic precipitator, and
that downstream of the electrostatic precipitator (13) the ozone-generating UV light device (7) is provided, which is designed to emit a constant amount of ozone to the cleaned air,
where the air purification process comprises:
sucking air into a treatment device,
removing ozone from the sucked-in air by UV radiation or catalysts and particles by the electrostatic precipitator and
before the cleaned air leaves the treatment device, generating a proportion of ozone and/or ionized gas from the cleaned air.

10. Air purification process according to claim 9,
wherein the proportion of ozone that is supplied to a space outside of a room of the treatment device can be switched on and off and its quantity can be controlled or regulated.

11. Air purification process according to claim 9,
wherein the ionization is conducted independently of the ozone generation.

12. Air purification process according to claim 9,
wherein an ion generator for the generation of ions from the cleaned air simultaneously generates regulated or controllable amounts of ozone.

13. Air purification process according to claim 9,
wherein either negative or positive ions are generated from the cleaned air.
